(19) United States
(12) Patent Application Publication
(10) Pub. No.: US 2005/0042240 A1
(43) Pub. Date: Feb. 24, 2005

(54) High viscosity antibacterials

(76) Inventors: David S. Utterberg, Seattle, WA (US); Fred G. Swindler, Bellevue, WA (US); Garrettson Ellis, Northfield, IL (US)

Correspondence Address: SEYFARTH SHAW LLP, Suite 4200, 55 East Monroe Street, Chicago, IL, 60603-5803, US

(21) Appl. No.: 10/901,949

(22) Filed: Jul. 29, 2004

Related U.S. Application Data
(63) Continuation-in-part of application No. 10/359,045, filed on Feb. 5, 2003.
Application No. 10/359,045 is a continuation-in-part of application No. 10/056,045, filed on Jan. 28, 2002, now abandoned.

(51) Int. Cl. A61K 31/045; A61K 9/00

(52) U.S. Cl. 424/400; 514/724

(57) ABSTRACT

An antibacterial fluid may be applied to a tubular medical cannula for access to a patient. The fluid comprises a typically metabolizable antibacterial formulation having a viscosity of at least about 5,000 cp. The cannula may then be inserted into the patient with an increased lubricity for a reduction of pain, while at the same time, unlike silicones, preferred materials do not readily accumulate in the patient. The tubular medical cannula may be a rigid, hollow needle, sharp or blunt, a spike, or a flexible catheter. Also, the viscous antibacterial fluid may be used to lock a catheter or other cannula while implanted in the patient, for storage purposes. The formulation is typically an alcohol plus a viscosity increasing agent.

### CROSS REFERENCE TO RELATED APPLICATION

This is a continuation-in-part of application Ser. No. 10/359,045, filed Feb. 5, 2003, which is a continuation-in-part of application Ser. No. 10/056,045, filed Jan. 28, 2002, now abandoned.

### BACKGROUND OF THE INVENTION

In the area of hemodialysis and other forms of therapy which require repeated access to the vascular system of a patient, the problem of vascular access remains significant, in large measure because of the problems with infection, and with clotting of blood in vascular access catheters.

One approach to the technical problem of effective, repeated vascular access involves the use of an implantable artificial port which is positioned under the skin of the patient. Then, a needle passes through the skin of the patient into the port to provide the vascular access.

Examples of such technology are illustrated by Finch et al. U.S. Pat. No. 5,562,617, Enegren et al. U.S. Pat. No. 4,955,861, and PCT International Publications WO97/47338; WO98/31416; and WO99/03527.

Needles which are used for access to the body may connect with such implanted ports, or they may connect with an arteriovenous fistula, or grafts, as is common in the art of hemodialysis and other extracorporeal blood therapies, or may cannulate any other body lumen or tissue, as in an intramuscular injection.

Such needles desirably have a silicone lubricant on their exterior surface to serve as a lubricant. This can significantly reduce the pain of the needle stick. However, silicone is not well metabolized, and is retained by the body. Thus, even though only tiny amounts of silicone enter into the patient with each needle stick, the amount of silicone can accumulate especially in patients who have lost their kidney function. Thus, there is a dilemma, in that to reduce patient pain it would be desirable to use a bit more silicone on the needle surface, while to reduce the accumulation of silicone in the patient, it is desirable to use little or no silicone.

Furthermore, silicone is not antibacterial in nature, i.e. it is neither bacteriostatic nor bactericidal.

Other attempts have been made to provide lubricating coating to needles. One of them, known as Spire coating is lubricating only after they have been hydrated. This takes a little time, and thus they are more useful for catheters which enter the body through previously made incisions than they are for cutting needles or other rigid cannulae.

Furthermore, needles may pass through the skin repeatedly through the same track (called a cannula or needle “tract” herein) so that they do not break through new tissue as they pass through the skin to engage an implanted port or body lumen such as an AV fistula. This needle tract, which represents a passageway through which fluids may flow and bacteria may pass, is desirably flushed, when part of an implanted port system, in reverse manner from the inner end of the needle tract to the outer end and through the skin, to remove bacteria which may have been drawn in by needle penetration or the like. However, current antibacterial flushing solutions have the additional disadvantage that they require time and expense to administer (e.g. by syringe and needle) and the effluent may dribble down the skin of the patient after coming out of the needle tract in an inconvenient and undesirable manner, since the dialysis position taken by patients is frequently semi-upright.

Further, typical topical disinfectants like isopropyl alcohol used in skin prep scrubs tend to evaporate before they can completely kill the bacteria they initially contact because only a film of alcohol adheres to the skin (with any excess running off). This rapid evaporation, which in the case of isopropyl alcohol is only seconds, also prevents these disinfectants from soaking down through detritus and dermal layers to where bacteria may dwell. Thus, only a low level of disinfection is achieved with typical topical disinfectants. Yet, these same agents maintained in liquid form are capable of very high levels of disinfection, for example when medical instruments are totally immersed in isopropyl alcohol. It would be advantageous for topical disinfection of injuries, cannulation sites or surgical sites if a means to retard the evaporative process for a volatile skin prep disinfectant were available.

It is also desirable to have anti-bacterial fluid surrounding the needle site during the procedure when the needle or other percutaneous device is penetrates through the skin and communicates with a body lumen, tissue or implanted port, so as to have an active disinfecting and/or physical barrier to block organisms from entering the annular tunnel between the cannula and the needle tract. Such antibacterial fluids generally need to be held within a gauze pad to prevent draining away from the needle or cannula tract site. However, the gauze provides increased wicking surface area, causing the antibacterial fluid to evaporate even more quickly than without the gauze. Evaporation stops the antibacterial action at the entrance to the cannula tract or the “tunnel.” Thus, it is necessary to be rather vigilant, repeatedly adding antibacterial fluid to the area around the outer entrance of the tunnel or needle tract.

Also, such needle tracts may be accidentally innoculated with bacteria due to bacteria alighting on an exposed needle, or otherwise being dragged in by the advancement of the needle through the needle tract from surrounding contaminated tissue or air. Conventional antibacterial fluids used to flush the needle tract or tunnel are of low viscosity, and thus migrate out of the tract and evaporate in fairly short order, causing the area between the needle and the needle tract to become a place where bacteria can grow. Additionally, conventional disinfectants such as alcohols are typically volatile at low temperatures, and thus evaporate quickly from their site of application before they have time to kill all microorganisms present.

Furthermore, there is a need to “lock” implanted catheters, by which is meant that an antithrombogenic solution such as heparin solution is placed into a catheter lumen which is implanted in the body, to suppress clotting as the blood migrates into the lumen of the catheter when it is not in use, such as between dialysis procedures. In the absence of such a catheter lock, substantial quantities of blood may migrate into the lumen of the catheter and clot there, rendering the implanted catheter useless.

However, because of the low viscosity of the typical antithrombogenic formulations containing heparin (and optionally antibacterial components such as alcohol or citric acid) the catheter lock solution diffuses away, and is replaced to a certain extent by blood during the period between dialyses, which may be on the order of 48 to 72 hours. Also, as the catheter lock solution diffuses slowly into the patient, its ingredients such as heparin, alcohol, citrate, citric acid, etc. get into the patient. This may result in certain toxic effects over the long run, since the catheter lock procedure is being used on a chronic basis between each dialysis procedure. For example, while isopropyl alcohol is a good antibacterial ingredient and is metabolizable, a study from Germany reports that toxic symptoms can arise with a daily dose exceeding only 500 mg of isopropyl alcohol.

Also, even conventional needles can be contaminated before use by exposure to air, for example when a particle of dust lands on the needle. This can be a source of unsterility when the needle enters the patient, or a needle or spike enters a sterile Y site, injection site or ampule.

The technical problems described above are reduced by the invention of this application, as described below.

### DESCRIPTION OF THE INVENTION

In accordance with one aspect of this invention, a low viscosity, relatively volatile topical antibacterial (antiseptic) agent such as isopropyl alcohol comprises an antibacterial (antiseptic) fluid or gel formulation having an elevated viscosity by a gelling agent. Preferably, the elevated viscosity may be about 5,000 to 150,000 centipoise (cp) when measured (in some embodiments 5,000 or 8,000 cp to 80,000 cp) and the gel may be self-supporting, essentially without flow characteristics at room temperature until it is disturbed. The higher viscosity inherently reduces the evaporation of common disinfectant agents such as isopropyl alcohol, ethyl alcohol, iodine, etc. As these topical agents cease disinfecting as soon as they evaporate away from the skin, by this invention we have simply yet greatly extended the disinfecting activity of these topical agents and hence their ability to impart higher levels of disinfection.

Preferably, more than a thin film of disinfecting agent can be applied to the skin, as the viscosity can render the gel self-supporting in much thicker quantities. Thus, this invention allows greater disinfection by the simple means of allowing a greater volume of disinfectant agent to be applied to the skin than physically possible by the low viscosity agent itself.

Preferably, the gelling agent comes out of solution and forms a surface film at the air interface of the gel drop as the alcohol begins to evaporate. This surface film further prevents evaporation of disinfecting agent within the film. Thus, by modification of the formulation herein, activity of the disinfectant agent on the skin may be maintained as long as for many hours.

In accordance with another aspect of this invention, an antibacterial (antiseptic) fluid or gel may be applied to a tubular medical cannula (that is, a needle, catheter, or tubular spike) for access to a patient or medical device communicating with a patient, where the fluid or gel comprises an antibacterial formulation having an elevated viscosity over aqueous solutions such as normal saline solution and povidone iodine. Preferably, the elevated viscosity may be about 5,000 to 150,000 centipoise (cp) when measured (in some embodiments 5000 or 8,000 cp to 80,000 cp), although a gel may be self-supporting, essentially without flow characteristics until it is disturbed. The viscosities stated herein are as measured by a Brookfield viscometer at 22° C. with an RV6 spindle at ten r.p.m. The cannula may be inserted into the patient. The word “antibacterial” throughout this document implies possible antiseptic effect against fungi also, and other microbes such as protozoa, i.e. antimicrobial.

The antibacterial fluid or gel may be applied by the manufacturer, the cannula being packaged to avoid evaporation. Otherwise, the fluid or gel may be applied by a nurse at the site of use by dipping the cannula into it or passing the cannula through the fluid or gel on the skin, for example.

The antibacterial fluid or gel may be placed on the outer wall of the cannula to serve as a lubricant for a sharp ended needle or a blunt ended cannula, for access to an implanted port, or alternatively to facilitate direct access by the cannula to a fistula or other blood vessel, body lumen or tissue of the patient. Preferably, the fluid or gel (hereafter generally called “fluid”) has a lubricating capability to reduce the friction of the cannula which is advancing into the patient, when compared with the same cannula advancement without the fluid. Generally, this lubricating effect is found spontaneously with the increased viscosity of the fluid formulae disclosed in this invention. In some uses, the viscosity of the antibacterial fluid of this invention may be about 10,000 to 50,000 cp. In other uses, the viscosity may be at least 50,000 cp; for example 70,000 to 100,000 cp. Also, the fluid evaporates less quickly, retaining antibacterial ingredients such as alcohols, for improved antibacterial effect.

The fluid of this invention may be placed on the cannula outer wall in an amount which is sufficient to cause some of the fluid to be wiped from the cannula upon said inserting of the cannula into the patient, so that a ring portion of the fluid visibly resides adjacent to the skin of the patient (or a medical device communicating with the patient). This provides a typically annular, antibacterial barrier pool at the outer end of a cannula tract that evaporates slowly, to suppress the entering and growth of bacteria and other microorganisms into the cannula tract. Alternatively, a small (such as a 1 cm tall by 1 or 2 cm. diameter) pool of the fluid may be placed on the skin at the cannula entry site, and the dry cannula may be passed into the skin through the pool. Thus, some of the fluid may preferably adhere to the cannula and pass into the needle tract, for antibacterial action there, while the pool provides an antibacterial seal at the needle entrance. The high viscosity fluid reduces the evaporation of alcohols and other antibacterial agents in it, greatly prolonging the antibacterial action. Preferred formulations are optically clear (transparent) enough to render the skin visible under the pool as the needle is inserted, especially when it is desired to penetrate a preformed needle tract or to visualize a blood vessel.

The antibacterial action of the exposed high viscosity fluid of this invention is particularly prolonged because of the tendency of preferred embodiments of the high viscosity fluid to form a skin on its exposed surface, which suppresses the diffusion of alcohol out of the material of this invention and prolongs the effect of alcohol content against the skin or another surface on which the high viscosity formulation resides.

Furthermore, preferred formulations of the high viscosity antiseptic material of this invention, pooled on the skin and surrounding the penetrating needle, tend to provide some adhesion to a needle or blunt cannulae and to the skin as the gelling agent dries and forms an intact film attaching to the cannula and to the patient's skin. Thus, some resistance to accidental withdrawal is provided by the use of preferred materials of this invention with needles that indwell the patient for a period of time, for example, dialysis fistula needles. Not only is antibacterial action provided, but a measure of protection against accidental falling out of an indwelling needle can also be provided by this invention. Such an accidental removal of a fistula needle during a dialysis process can have serious consequences if the fall-out of the needle is not noticed, for example during nocturnal dialysis.

Additionally, cannulation pain can be reduced if the patient's cannulation site is cooled prior to cannulation. Many pheresis clinics rub the cannulation site with an ice cube prior to inserting the cannula through the patient's skin. Also, ethyl acetate is sprayed on cannulation sites and other painful areas, and its intensive evaporation rapidly cools the skin and underlying tissue, and reduces the pain.

Formulations of this invention may be cooled down to a freezing temperature such as −5 to +5 degrees C., and its disinfectant ingredients may remain fluid for minutes to hours longer than if applied at room temperature. When applied to the skin, the volume of gelled antiseptic intensively cools the skin directly in contact, staying in position. The cooling effect is superior to cooled liquid alcohol, films of which rapidly evaporate with less heat absorption from the skin. Thus, greater pain relief may be obtained from use of this invention as a topical disinfectant prior to cannulation.

Typically, the antibacterial fluid of this invention comprises a low viscosity antibacterial agent mixed with a viscosity increasing agent. Examples of antibacterial agents which may be used comprise alcohols, chlorhexidine, Chlorpactin, iodine, tauroline, citric acid, sodium hypochlorite, soluble citric acid salts, particularly sodium citrate, optionally mixed with water, and alkali metal salts of ethylene diaminetetraacetic acid (EDTA) such as tetrasodium EDTA, as described in WO 03/047341.

Examples of viscosity increasing agents comprise Carbopol, starch, methylcellulose, carboxypolymethylene, carboxymethyl cellulose, hydroxypropylcellulose, or the like, preferably a material such as starch which can clear out of the body of the patient by metabolization or excretion in the quantities used, so that the material does not accumulate in the body, long term. This property is defined herein by the phrase “body clearing”. Carbopol is a cross-linked polyacrylic acid based polymer sold by Noveon, Inc. It is preferably neutralized to about pH7 with a base material such as tetrahydroxypropyl ethylene diamine, triethanolamine, or sodium hydroxide. Derivatives of starch may also be used, such as hydroxyethylstarch, hydroxypropylstarch, or starch having bonded organic acid ester groups, to improve compatibility with antibacterial agents such as alcohols, for example ethanol or isopropanol. Such ester groups may be the reaction product of two to twelve carbon organic acids with the starch, for example. Also, the elevated viscosity antiseptic fluid may be created by the use of a fat emulsion, or other dispersions in water/alcohol of glycerol mono or di esters of fatty acids, or fatty acid esters of other polyols such as sugars having one or more bonded fatty acid groups per molecule. Analogous compounds with ether linkages may also be used.

Also, other materials such as alginic acid, with or without calcium citrate may be used as viscosity increasers, or polyvinyl alcohol (with or without borax) povidone, polyvinylpyrrolidone, polyethylene glycol alginate, sodium alginate, chitosan, and/or tragacanth. Polyvinylpyrrolidone can be body clearing.

If desired, crosslinking agents may be applied, for example polyethyleneimine, which is capable of crosslinking polyvinylpyrrolidone in an alcohol-water mixture to create a gel-like formulation. Other crosslinking agents may be used in a manner known to those skilled in the art, for example glutaraldehyde or acetic anhydride. Other crosslinking agents may comprise e-beam radiation, gamma radiation, sulfuric acid, various acrylate compounds, calcium pantothenate, aspartic acid, glutamic acid, sodium borate or various sulfate and phosphate compounds, used in a manner appropriate to the particular chemistry of the crosslinking agent used.

The material of this invention may contain other agents as well, for example, clotting (hemostatic) agents such as collagen and other known materials, including simple inorganic salts and other promoters for blood clotting particularly outside of the body. Other examples of possible clotting agents include:

1. Aluminum Ammonium Sulfate

2. Aluminum Potassium Sulfate

3. Chitosan

4. Epinephrine, (1:50,000-1:1,000)

5. Tannic Acid

6. Collagen

7. Styptic Collodion

8. Hyaluronic Acid

9. Sodium Hyaluronate

10. Aluminum Sulfate

11. Cotarine

12. Cotarine Chloride

13. Cotarninium Chloride

These ingredients may be admixed to form the fluid of this invention at any desired elevated viscosity, for the purpose of achieving the advantages of this invention by reducing the disadvantages discussed above, while also providing needle lubrication when desired. If desired, the fluid of this invention may also contain an effective amount of an antithrombogenic agent such as heparin, and a diluent such as water, along with other desired ingredients.

Alternatively, or additionally, the fluid of this invention may be applied to the lumen of a cannula such as a catheter, to provide a lock that restricts the flowing of body fluids into the cannula. Also, the fluid of this invention may be used with any cannula, spike, catheter, or the like for any purpose, to provide a retentive, self-sterilizing characteristic to the product.

In one embodiment, the formulation of this invention may comprise a mixture of isopropyl alcohol and neutralized Carbopol, with other optional ingredients being present such as water, antithrombogenic agents such as heparin, and the like. Preferably, about 0.4 to 2 weight percent of Carbopol is present. Citric acid may also be present as an antibacterial agent, either with or as a substitute for another anti-bacterial agent such as isopropyl alcohol or ethanol.

In another embodiment, a gel of isopropyl alcohol, optionally with up to about 30 weight percent water, may be formed with 2.2 weight percent hydroxypropylcellulose, to form a high viscosity antibacterial agent of this invention.

In a third embodiment, a gel may be prepared from a mixture of about 60-80 volume (v/v) percent of ethyl or isopropyl alcohol having 1 to 20 weight percent (w/w) of polyvinylpyrrolidone, which is generally a body-cleaning agent, for example having a molecular weight of about 44,000; from 0.1 to 2 weight percent (w/w) of polyethyleneimine as a crosslinking agent; and, optionally, up to about 10 percent (w/w) of polyethylene glycol, for example of a molecular weight of about 400. About 20 to 40 volume percent of water may be added to make up 100 volume percent, to provide a generally optically clear gel material.

Similar gel formulations may be prepared where the polyethyleneimine crosslinking agent is replaced by another known crosslinking agent such as 0.5 to 10 weight percent (w/w) of glutaraldehyde or 0.5 to 10 weight percent (w/w) of acetic anhydride.

Such formulations provide antiseptic gel materials which may be used in the various ways described above.

Another class of gel formulations may comprise 60 to 80 per volume percent (v/v) of isopropyl or ethyl alcohol; from 0.2 to 6 weight percent (w/w) of chitosan; from 0.1 to 6 weight percent of a polyvinylpyrrolidone having the approximate molecular weight previously used; optionally up to 10 weight percent (w/w) of polyethylene glycol of similar molecular weight to that used previously, and water to make up 100 volume percent.

As before, glutaraldehyde; acetic anhydride, or polyethyleneimine may be used typically in the concentrations previously specified as crosslinking agents in this formulation, if desired.

Antiseptic gels of the type created by this type of formulation may also be used in the ways previously described above.

The antibacterial, viscous fluid of this invention may be provided to the user in an inexpensive squeeze-delivery container, to avoid the need for a syringe or other more expensive delivery system. A squeeze-delivery container may be a one piece, blow molded container in which the contents are administered by simple manual squeezing of the fingers. Specifically, the squeeze-delivery container which holds the antibacterial fluid of this invention may carry a male luer typically having an inner diameter at its tip of least about 2 millimeters. One may attach the male luer of the container to a female luer of a rigid cannula or catheter, which may be emplaced in the body of a patient. One then squeezes the container for a simple transfer of the antibacterial formulation into the rigid cannula or catheter.

Further in accordance with this invention, one may flush a preferably metabolizable, antibacterial fluid through a cannula tract which extends through the skin of a patient and inwardly therefrom. The method comprises the steps of inserting a cannula into the cannula tract; and passing the fluid through the cannula to exit the cannula at an inner portion of the tract, and to cause the fluid to flow outwardly through the tract outside of the cannula so that some of the fluid exits around the cannula through the skin, where some of it is retained. The antibacterial fluid may have, for example a viscosity of about 10,000-30,000 cp, or typically up to about 50,000 cp or even 100,000 cp, and it may be a formulation similar to that previously described. The cannula tract may communicate its inner end with an implanted, artificial port, which communicates with a body lumen of a patient.

Furthermore by this invention, one may place a preferably metabolizable fluid into a lumen of a catheter installed in a patient, typically a permanently implanted catheter, to “lock” the catheter, reducing the migration of body fluids into the catheter lumen while the catheter is not in use, to thus avoid clotting as the catheter resides in the patient. The fluid may have for example a viscosity of about 10,000-50,000 cp, and may be a fluid as previously described. Such fluids may comprise an antibacterial agent such as alcohol and/or an antithrombogenic agent. Also, the alcohol content may be reduced in the high viscosity formulation, diluted with water down to a concentration as low as 10 vol. %, because the formulation will usually have a multihour or multiday dwell time in the catheter, permitting a slow microorganism kill, and the risk is reduced if the material is accidentally infused into the blood stream.

This “lock” can be better achieved because of the increased viscosity of the fluid in accordance with this invention, which thus physically resists removal from the lumen of the catheter and replacement by blood while residing in the body between uses of the catheter. Also, as previously taught, there may be present an antibacterial agent and/or an antithrombogenic agent which similarly is physically prevented by the gelling agent from easily diffusing into the bloodstream. For example, a gelled heparin solution at a suitable concentration may be used, exhibiting the elevated viscosity on testing of preferably about 5,000-80,000 cp, when measured, so that any blood that does enter into the lumen is going to encounter conditions where clotting is suppressed because of the presence of heparin, and microbial growth may be suppressed when an antibacterial agent is present.

As another alternative, a gelled dispersion of an antibiotic, such as finely divided silver, or a biologically derived antibiotic such as Gentamycin, with or without alcohol present, can be used as a viscous catheter lock material. A gelled aqueous dispersion of an antibiotic of any desired type tends to be retained in the catheter, for the most part, and not distributed into the blood stream, so that the microorganism suppression that the antibiotic provides is localized. This also reduces the possibility of creating drug resistant bacteria in the body because the antibiotic tends to remain localized within the catheter, and thus stays in high concentration. An effective dose of any antibiotic may be carried in an aqueous fluid of increased viscosity in accordance with this invention, for example, about 10,000-50,000 cp., and used for catheter locking.

Also, by this invention, a preferably body clearing, antibacterial fluid described above can be used to coat hypodermic needles, spikes or the like to reduce needle contamination, since the needle or spike comprise an actively disinfecting surface film. Simultaneously, the fluid material of this invention may be used as a desirable needle lubricant, but providing active sterility so that dust particles that land on the needle when the needle is exposed to the air, or other contamination, tend to be sterilized so that the contamination does not spread to the patient, or to a sterile Y site, ampule, or the like.

Additionally, the formulations of this invention may be squeezed out onto the skin, especially when gel-like in consistency, for example at a viscosity of about 20,000 to 50,000 cp, or up to about 100,000 cp, to form a little sterilizing pool on the skin. The gel retards the evaporation of the disinfecting medium, thus giving greater “contact time” of said medium with any infecting agent it encounters on the skin. Additionally, the high viscosity retards the movement of the pool by gravity or patient movement. Then, a needle may pass through the viscous material of this invention, to provide further assurance of sterile entry of the needle and subsequent protection along the needle and at the skin entry point with less evaporation of antiseptic than with current techniques. This may be used with fistula needles in hemodialysis and the like, with good needle lubrication being provided by the pool material for reduced pain.

Also, the formulations of this invention may be placed on the skin or other body surface of a patient such as fingernails or toenails for the purpose of eliminating or retarding present disease caused by microorganisms. Examples of the such disease might be eczema, ringworm, skin infection by another fungus or bacteria, or a fungal infection of the toenails. It is believed that low viscosity antimicrobial agents, particularly isopropanol, can be effective against stubborn microorganisms, but for the fact that they are volatile, so that the contact time is low. By this invention, the contact time of isopropanol or the like can be very substantially extended, significantly increasing the antimicrobial effect of isopropanol or similar volatile, antimicrobial agents. Also, low molecular weight antimicrobial agents such as isopropanol are believed to have good penetrating capability. For example, it is believed that they can penetrate through the toenails, for example to get into contact with fungal toenail infections, to reduce or eliminate the infection.

Preferably, the viscosity of the formulation used is at least about 50,000 cp, to minimize evaporation of isopropanol, or another volatile antimicrobial formulation, that may be carried into contact with an infected body surface by means of one of the formulations of this invention.

### DESCRIPTION OF DRAWINGS

Referring to the drawings, FIG. 1 is a vertical section of a tubular medical cannula, shown to be penetrating the skin of the patient and connecting with an implanted artificial port, which is shown in schematic form.

FIG. 2 is an elevational view of a catheter which is implanted to extend through the skin of the patient and to connect with an implanted artificial port, with the catheter being releasably connected with a container of the antibacterial fluid of this invention.

FIG. 3 is a schematic view of separated components of a medical kit, the components being for practicing methods of this invention.

### DESCRIPTION OF SPECIFIC EMBODIMENTS

Referring to FIG. 1, an angled cannula 10 is shown to be penetrating the skin 12 of a patient, to extend along a cannula or needle tract 16 through tissue of the patient to enter into sealing, flow communication with a port 14, implanted within the tissue of the patient under the skin 12. Broadly speaking, the technique is similar to that discussed in the PCT publications WO98/31416 and WO99/03527, as cited above. Conduit 15 is connected to a blood vessel of the patient. A known valve is present to control flow through conduit 15.

Cannula set 10 carries a rigid cannula 18 which may either have a sharp tip or a blunt tip 20, to provide communication through the skin 12 between the implanted port 14 and a flow conduit 22, which may comprise a conduit through cannula member 10 as shown, which conduit may also extend into the lumen of connected, flexible tubing 24. A suitable resealable plug 26 may be provided, carrying a preformed slit if desired, to provide needle access to the flow conduit through resealable plug 26, as previously disclosed in Utterberg et al. U.S. Pat. No. 6,267,750, entitled Tapered Intravenous Cannula. As disclosed there, cannula 18 may also be tapered and blunt, if desired.

In accordance with this invention, cannula 18 may be inserted into cannula or needle tract 16, which may be a preformed tract created by previous cannula penetrations so that the preferably blunt cannula 18 does not cut through tissue which has not been previously cut by prior penetrations of cannula needles, to facilitate the penetration of cannula 18 into needle tract 16 without pain.

An antibacterial fluid having a lubricating capacity may be provided to the outer surface of cannula 18, to reduce the friction of cannula 18 advancing into the patient. For example this fluid has a viscosity of about 25,000 cp. This antibacterial fluid may comprise an aqueous solution of about 50 to 90 weight percent of ethyl alcohol or isopropyl alcohol, from zero to 10 weight percent of dissolved citric acid, and sufficient viscosity increasing agent, particularly neutralized Carbopol, hydroxypropylcellulose, or a starch derivative, to provide the desired viscosity to the aqueous solution. Typically, about 10 to 40 weight percent of water will be present. Typically, from 0.4 to 0.7 weight percent of Carbopol may be used, or from 2 to 4 weight percent of hydroxypropylcellulose.

For example, specific formulations may comprise an aqueous isopropyl alcohol solution (70% alcohol and 30% water) containing 0.5 weight percent of neutralized Carbopol, or 2.2 weight percent of hydroxypropylcellulose, to provide a viscous, gel-like material.

A sufficient amount of the fluid of this invention may be placed on the outer wall of cannula 18 so that, as cannula 18 advances through cannula tract 16, some of the fluid is wiped from the cannula and visibly resides in the annular junction 26 between the cannula 18 and the skin 12, to serve as an antiseptic reservoir at the outer end of needle tract 16, thus protecting the tubular opening defined by needle tract 16 between cannula 18 and the wall of needle tract 16. Alternatively, one may place a small portion of the viscous, gel-like fluid 27 on the skin over needle tract 16, passing cannula 18 through it into needle tract 16. Thus the pool of fluid 27 forms a continuing antibacterial seal that holds its antiseptic such as alcohol with less evaporation, for better antibacterial action. Hydroxypropyl cellulose serves well to provide a suitable, stable, gel-like emulsion.

If desired, an effective amount of an antithrombogenic agent such as heparin may also be added to the antibacterial fluid of this invention.

The typical purpose of the connection of cannula member 10 and implanted port 14 is to provide access for extracorporeal blood transport between the vascular system of the patient and an extracorporeal blood processing device such as a hemodialyzer. Two of such connections of the type as shown in FIG. 1 may be typically used in a hemodialysis process, with the blood passing into cannula 18 from port 14, which connects with a vein of the patient. The blood then passes through tubing 24 to a dialyzer or other blood treatment device, and then is correspondingly returned through another, similar connection.

Alternatively, about 1 to 4 weight percent of ethylcellulose, hydroxyethylstarch, or hydroxypropylstarch may be used as the viscosity increasing agent.

A currently proposed formulation for the gelled antibacterial fluid of this invention may comprise about 70 volume percent (v/v) of isopropyl alcohol, 10 wt. percent (w/w) of polyvinylpyrrolidone of about 1,500,000 Daltons; 1 wt. percent (w/w) of polyethyleneimine, the balance comprising water. The polyethylene glycol improves handling properties of the gel product.

Another currently proposed formulation comprises about 70 percent (v/v) isopropyl alcohol; 3 weight percent (w/w) of chitosan; 3 weight percent of (w/w) of polyvinylpyrrolidone; and 5 weight percent (w/w) of polyethylene glycol (6,000 Daltons), the balance comprising water. The polyethylene glycol improves handling properties of the gel product.

Such formulations may, for example, have a viscosity of about 10,000 cp to about 80,000 cp, for example about 35,000 cp.

Another currently proposed formulation comprises about 65-75 volume percent, (v/v) of 99% isopropyl alcohol; 4-7 weight percent (w/w) of polyvinylpyrrolidone c-30 (for example 5.5%); from 0.6-1 weight percent (w/w) of polyethyleneimine; from 4-6 weight percent (w/w) of propylene glycol; and from 4-6 weight percent (w/w) of polyethylene glycol (8000 Daltons). Purified water is then added to make up the 100% balance.

For example, a specific formulation may contain 0.78 liter of the 99% isopropyl alcohol; 55 grams of the polyvinylpyrrolidone; 8.25 grams of the polyethyleneimine; 50 grams of propylene glycol, 50 grams of polyethylene glycol (8000 Daltons), and sufficient purified water to make one kilogram of preferred material in accordance with this invention. After mixing, the viscosity of a such a material is about 85,000 cp. The material can be used in the various uses described in this invention. Also, it may be used as a skin antiseptic generally, with the volatile antiseptic being retained in contact with the skin for an extended period of time, compared with the simple application of an antiseptic in substantially pure form, to combat skin infections.

As another example, a formulation in accordance with this invention may be made by providing 700 grams of 99% isopropanol into a vessel for mixing. A stirrer is used to form a vortex. Then, 50 grams of propylene glycol are added (the material serving to improve the properties of the gel). Following this, 55 grams of polyvinylpyrrolidone C-30, K-30 is added, with stirring, until no lumps are observed and all powder is dissolved.

One hundred grams of sterile water is placed into another container, and 50 grams of polyethylene glycol 8000 is added, to improve gel properties, with stirring until the polymer is dissolved in the water. This solution is then added to the isopropanol-propylene glycol solution, with stirring.

Following this 16.5 grams of polyethyleneimine (50%) is blended with 28.5 grams of sterile water, this dispersion then being added to the solution previously prepared, while stirring slowly, to add the diluted polyethyleneimine solution. Stirring is continued for about 10 minutes, followed by transfer of the solution to a closed, alcohol-impermeable container for storage. The viscosity of the material increases over a period of about 3-5 days to form a gel having a viscosity of about 85,000 cp.

Further in accordance with this invention, after cannula 18 has been inserted into needle or cannula tract 16 as shown in FIG. 1, extending through the skin of the patient, preferably a metabolizable, antibacterial fluid in accordance with this preferred embodiment is passed through cannula 18 inwardly, to exit the cannula at end 20. As is known, implantable port 14 may have a valve so that the antibacterial fluid from cannula 18 cannot pass further into port 14, but rather, the fluid then flows outwardly through tract 16, outside of cannula 18, to flush cannula tract 16 in a known manner (but for the composition of the antibacterial fluid of this invention,) taking with it bacteria and other contamination to reduce infection. By way of advantage, the fluid has an increased viscosity of at least 5,000 or 10,000 cp, and preferably about 20,000-50,000 cp, or even up to about 100,000 cp, so as to be able to flush cannula tract 16, while being immobile enough through its elevated viscosity to resist migration out of the tract 16, and away from annular junction 26, when positive flushing is not taking place. Thus, better antibacterial effect may be provided while cannula 18 resides in cannula tract 16.

It also may be desirable to allow the fluid of this invention to reside in the lumen of cannula 18 to serve as a “lock”, i.e. a protection against the migration of stagnant blood into the cannula while it is not being used, to prevent against clotting of blood and bacteria build up within the cannula, and to reduce chances of forming a biofilm that can reduce flow through cannula 18.

If desired, the antibacterial fluid of this invention may be administered by a syringe or other container through resealable needle access plug 26. Also, when desired, such antibacterial fluid can be removed from cannula 18 in a similar manner, when it is undesirable to commingle the entire aliquot of antibacterial fluid with blood or other fluid normally transported through the system during use. The viscous fluid is better retained in a cannula or catheter, particularly at viscosities of 10,000 cp or higher.

The fluid used is also preferably antimicrobial in nature, to prevent the growth of bacteria, fungi, and other microorganisms.

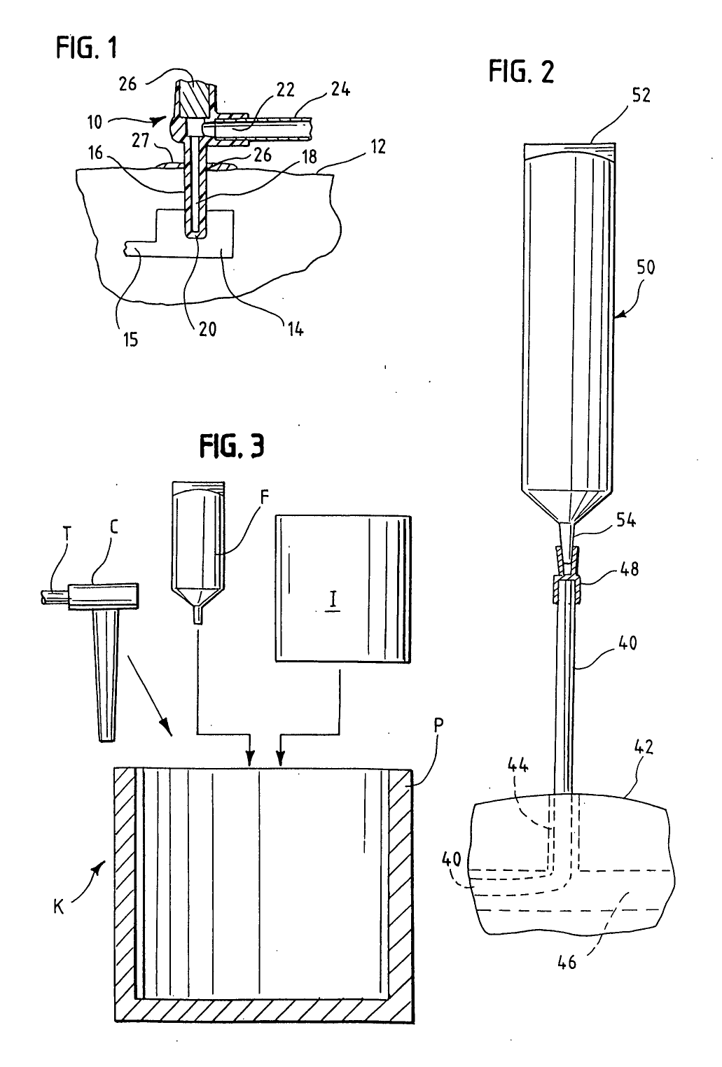

Referring to FIG. 2, another type of use of the antibacterial fluid of this invention is shown. An implanted catheter 40 is shown extending inwardly through the skin 42 of a patient, passing through a tissue tunnel 44 and being sutured into communication with a vein 46 of the patient for obtaining blood access to the patient, for extracorporeal blood processing such as hemodialysis. Often, two such implanted catheters are provided to a patient.

Catheter 40 terminates in a female luer connector 48. By this invention, a squeeze-delivery container 50, containing the antibacterial fluid of this invention, is provided. Container 50 may comprise a blow molded container, or a length of flexible tubing sealed at its upper end 52, and carrying an integral male luer connector 54 at its lower end, capable of releasable sealing engagement with female luer connector 48. Preferably, male luer 54 has a lumen with an inner diameter of at least 2 mm.

Thus, after attachment of container 50, which holds the viscous fluid of this invention, one may squeeze container 50 between uses of catheter 40 to substantially fill catheter 40 with the viscous fluid of this invention, thus providing a “catheter lock”. In one embodiment, the fluid viscosity may be about 30,000 to 40,000 cp. This lock suppresses the migration of blood into catheter 40, where the blood can clot and block flow in the catheter. Also, microorganism growth within catheter 40 is reduced, as well as the formation of biofilms, which can eliminate catheter usefulness by blocking blood diffusion flow into the catheter. Because of the increased viscosity of the antibacterial fluid of this invention, it is more effective as a catheter lock than known solutions, lasting for several days while reducing the migration of blood into the catheter lumen during storage.

When it is desired to open the catheter again for extracorporeal blood flow, the fluid of this invention filling catheter 40 during the catheter lock period is optionally removed by a syringe or the like through connector 48, so that most of the antibacterial fluid is not mixed with blood of the patient. However, those amounts of the antibacterial fluid which are mixed can readily be cleared by the body with proper selection of ingredients in accordance with this invention.

Here also it may be desirable to incorporate an antithrombogenic agent such as heparin into the antibacterial fluid in an effective concentration, to suppress the clotting of any blood that does find its way into catheter 40 during the catheter lock period.

Referring to FIG. 3, a kit is shown in exploded condition for practicing the various methods of this invention. A set comprising a length of tubing T, connected to a tubular medical cannula C for access to the patient, is provided. Alternatively, cannula C may comprise a catheter for connection with the blood supply of a patient, if desired. Alternatively, element C and connected tubing T may be eliminated from kit K.

Kit K also contains a fluid container F of the fluid of this invention, for application either to a catheter or a rigid cannula. Packaging unit P is also provided to contain the various elements of the kit, the packaging unit P being a sealable envelope, typically capable of gas sterilization, or a tray with a porous cover having similar sterilization capability, or the like.

Instructions I are also included, providing instructions on the use of the fluid F of this invention in conjunction with cannula or catheter C in accordance with any of the previously described methods for applying antibacterial fluid to a medical cannula such as a rigid needle, a flexible catheter, or the like, as previously described.

Preferably, because of increased viscosity, the antibacterial fluid of this invention significantly reduces the friction of a needle or other cannula as it is advanced into the patient, typically a catheter, a fistula needle, or a cannula entering through a cannula or needle tract. The fluids of this invention are instantly lubricious, and do not require a hydration step, as is the case for some catheter lubricants. There can be antibacterial characteristics, which provide significant advantage over such hydratable materials and silicones. The preferred fluids of this invention also are retained more persistently on the skin in the vicinity of a catheter or rigid cannula within the patient because of the increased viscosity, resulting in the significant advantage of better antibacterial effect. Also, they are less likely to evaporate or dribble away from the needle or cannula tract along the skin. The fluid of this invention may coat the interior walls of a catheter, with the bulk fluid being removed. The increased viscosity of the fluid can create such a coating, to durably act as an antimicrobial agent without the presence of the bulk fluid filling the catheter or other cannula.

Medical needles of any type may have their surfaces liberally applied as described above with the viscous, antibacterial fluid of this invention for increased comfort to a patient, while the needle retains a self-sterilizing characteristic as the needle is inserted, with less concern about the accumulation of materials from the fluid in the patient over the long term. Fistula needles for dialysis may be so coated, retaining better sterility as they are exposed to the air during the priming process.

The above has been offered for illustrative purposes only, and is not intended to limit the scope of the invention of this application, which is as defined in the claims below.

1. An antibacterial formulation which comprises an alcohol mixed with sufficient viscosity increasing agent to provide a viscosity of about 5,000 to 150,000 cp to the formulation, said formulation being substantially free of antibacterial agents other than said alcohol.

2. The formulation of claim 1 in which said alcohol comprises ethanol or isopropanol.

3. The formulation of claim 1 which is transparent.

4. The formulation of claim 1 in which said viscosity of the formulation is from 70,000 to 100,000 cp.

5. The formulation of claim 1 in which said viscosity increasing agent comprises crosslinked polyvinylpyrrolidone.

6. The formulation of claim 5 which has a viscosity of about 85,000 cp.

7. The formulation of claim 1 which is body-clearing.

8. The formulation of claim 1 in which said viscosity increasing agent comprises chitosan and polyvinylpyrrolidone.

9. A cannula, carried by a hub and having a surface at least partially coated with the formulation of claim 1.

10. The method which comprises placing a fluid on the outer surface of a medical cannula, said fluid comprising an antimicrobial agent and having a viscosity of about 5,000 to 150,000 cp, and thereafter inserting the cannula through the skin of the patient or into a sterile receptacle.

11. The method which comprises placing a portion of a fluid, which comprises an antimicrobial agent and has a viscosity of about 5,000 cp to 150,000 cp, on the skin of a patient to form a fluid layer on the skin, and thereafter passing a medical cannula through the fluid layer on the skin and through the skin of the patient.

12. The method of claim 11 in which said medical cannula is carried by a hub and connects with flexible tubing, said cannula extending transversely to the axis of said flexible tubing adjacent to said hub.

13. The formulation of claim 1 in which said viscosity increasing agent comprises polyvinylpyrrolidone.

14. The formulation of claim 1 in which a clotting agent is also present.

15. A formulation which comprises a relatively low viscosity, volatile antibacterial agent mixed with sufficient viscosity increasing agent to provide a viscosity of about 5,000 to 150,000 cp., said formulation having the characteristic of forming a skin on its surface when exposed to the air, to retard the evaporation of said volatile antimicrobial agent, and thus to prolong its antibacterial effect when placed in contact with the skin or other surface.

16. The formulation of claim 15 in which said viscosity increasing agent comprises polyvinylpyrrolidone crosslinked with polyethyleneimine.

17. The formulation of claim 16 which has a viscosity of essentially 70,000 to 100,000 cp.

18. The formulation of claim 17 in which said antimicrobial agent comprises alcohol.

19. A medical cannula which extends through the skin of a patient and which connects to a medical flow conduit at an end of said cannula which is outside of the skin, said medical cannula extending through a pool of antimicrobial formulation carried on the skin of the patient, said pool comprising a relatively low viscosity antimicrobial agent mixed with sufficient viscosity increasing agent to provide a viscosity of about 5,000 to 150,000 cp, portions of said medical cannula being coated with said antimicrobial formulation.

20. The cannula of claim 19 in which said antimicrobial agent is isopropanol.

21. The cannula of claim 19 in which said viscosity increasing agent is polyvinylpyrrolidone crosslinked with polyethyleneimine.

22. The cannula of claim 19 in which said pool of antimicrobial formulation also serves as a removable adhesive to help retain said cannula in position.

23. The method which comprises:
placing on a body surface of a patient an antimicrobial formulation which consists essentially of an alcohol mixed with sufficient viscosity increasing agent to provide a viscosity of about 5,000 to 150,000 cp to the formulation.

24. The method of claim 23 in which the viscosity of the formulation is at least about 50,000 cp.

25. The method of claim 23 in which the alcohol comprises ethanol or isopropanol.

26. The method of claim 23 in which the alcohol comprises isopropanol.

27. The method of claim 23 in which the viscosity increasing agent comprises crosslinked polyvinylpyrrolidone.

28. The method of claim 23 in which said antimicrobial formulation is placed on a patient's body surface that is infected with a microorganism.

29. The method of claim 28 in which the antimicrobial formulation remains on the body surface until essentially all of the alcohol has dissipated from the formulation.

30. The method of claim 28 in which the antimicrobial formulation is placed on the skin of patient.

31. A gel material which consists essentially of at least 50 wt. percent of an alcohol plus a gelling agent.

32. The gel of claim 31 in which said alcohol is isopropanol.

33. The gel of claim 31 which is generally optically clear.

34. The gel of claim 31 in which said gelling agent comprises crosslinked polyvinylpyrrolidone.

35. The gel of claim 31 which has a viscosity of essentially 70,000 to 100,000 cp.

36. The gel of claim 31 which further contains a blood clotting agent.

37. An antibacterial formulation which comprises 65-75 volume percent of isopropyl alcohol; 4-7 wt. percent of polyvinylprrolidone; from 0.6-1 wt. percent of polyethyleneimine, and water to make up a 100 percent balance.

38. The antibacterial formulation of claim 37 which further comprises 4-6 wt. percent propylene glycol and from 4-6 wt. percent of polyethylene glycol.

39. The formulation of claim 37 which is substantially optically clear.

40. An antibacterial formulation which comprises a mixture of 60-80 volume percent of isopropanol alcohol or ethyl alcohol; 1-20 wt. percent of polyvinylpyrrolidone; from 0.1-2 wt. percent of a crosslinking agent, and the balance comprising water, said formulation comprising a gel having a viscosity of about 50,000 to 100,000 cp.

41. The formulation of claim 40 in which said crosslinking agent is selected from the group consisting of 0.1-2 wt. percent of polyethyleneimine; 0.5-10 wt. percent of glutaraldehyde, and 0.5-10 wt. percent of acetic anhydride.

42. The formulation of claim 40 which is substantially optically clear.

43. A gel formulation which comprises 60-80 volume percent of isopropyl or ethyl alcohol; from 0.2-6 wt. percent of chitosan, from 0.1-6 wt. percent of polyvinylpyrrolidone; from 0-10 wt. percent of polyethylene glycol, and water to make up 100 volume percent.

44. The gel formulation of claim 43 having a viscosity of essentially 50,000 to 100,000 cp.

45. The antibacterial formulation of claim 1 in which said viscosity increasing agent is a starch.

46. A formulation which comprises an antibiotic mixed with sufficient viscosity increasing agent and water to provide a viscosity of about 5,000 to 150,000 cp to the formulation.

47. The formulation of claim 46 in which the viscosity increasing agent comprises crosslinked polyvinylpyrrolidone.

48. The formulation of claim 46 which comprises a self-supporting gel.

49. A catheter having a lumen which is at least partially filled with the formulation of claim 46.

50. The method of claim 46 in which said antibiotic comprises a biologically derived antibiotic.

51. The method which comprises:
placing a fluid into a lumen of a catheter installed in a patient to “lock” the catheter, to reduce the flow of body fluids into the catheter lumen as the catheter resides in the patient, said fluid having a viscosity of about 5,000 to 150,000 cp and containing an antibiotic.

52. The method of 51 in which said antibiotic comprises a biologically derived antibiotic.

53. The method of claim 51 in which said antibiotic is Gentamycin.
